(19) United States
(12) Plant Patent Application Publication
(10) Pub. No.: US 2012/0180173 P1
(43) Pub. Date: Jul. 12, 2012

(54) Variety of lantana plant named 'NEBLAN5151'

(50) ;

(75) Inventor: Hans de Does, Muenster (DE)

(73) Assignee: Volmary GmbH, Muenster (DE)

(21) Appl. No.: 13/135,185

(22) Filed: Jun. 28, 2011

Related U.S. Application Data
(60) Provisional application No. 61/460,911, filed on Jan. 10, 2011.

(51) Int. Cl. A01H 5/00

(52) U.S. Cl. PLT/227

(57) ABSTRACT

‘NEBLAN5151’ is a new variety of lantana plant having a compact growth habit and white and lemon bicolored flowers.

### CROSS REFERENCE TO RELATED APPLICATION

This application claims the benefit of U.S. Provisional Patent Application Ser. No. 61/460,911, filed on Jan. 10, 2011, and entitled “Variety of Lantana Plant named ‘NEBLAN5151’”, the entire disclosure of which is incorporated herein by reference.

### BOTANICAL CLASSIFICATION

Lantana camara

### VARIETAL DENOMINATION

‘NEBLAN5151’

### BACKGROUND OF THE INVENTION

The present invention comprises a new and distinct cultivar of Lantana camara known by the varietal name ‘NEBLAN5151’. The new variety was discovered in Muenster, Germany in the Summer of 2007. The new variety is a result of a cross of proprietary and unpatented lantana plants. The purpose of the breeding program was to develop a lantana variety that was early flowering, with a compact growth habit and low seed setting. The new variety is similar in plant habit and flower size to its female parent, but differs in flower color. ‘NEBLAN5151’ is similar in plant habit to its male parent, but differs in flower color. Further, the new variety is similar in plant habit and early flowering time to lantana variety ‘Lucky White’ (unpatented), but differs in flower color, as ‘Lucky White’ has all white flowers, and the present variety has white flowers with lemon-colored centers. The new variety was first asexually reproduced by cuttings in September of 2007 in Muenster, Germany. The new variety has been trial and field tested and has been found to retain its distinctive characteristics and remain true to type through successive propagations.

The following characteristics have been repeatedly observed and are determined to be unique characteristics that distinguish ‘NEBLAN5151’ as a new and distinct cultivar from others known to the breeder:

1) Very compact plant habit;

2) Good branching with short internodes;

3) Early flowering time that is 2 weeks earlier than lantana standards such as ‘Goldsonne’ (unpatented); and

4) Small to medium-sized bicolored flowers (bright lemon and white).

### DESCRIPTION OF THE DRAWINGS

The following photographic drawings illustrate the new variety, with the color being as nearly true as is possible with color illustrations of this type:

FIG. 1 is a photograph of the new variety; and

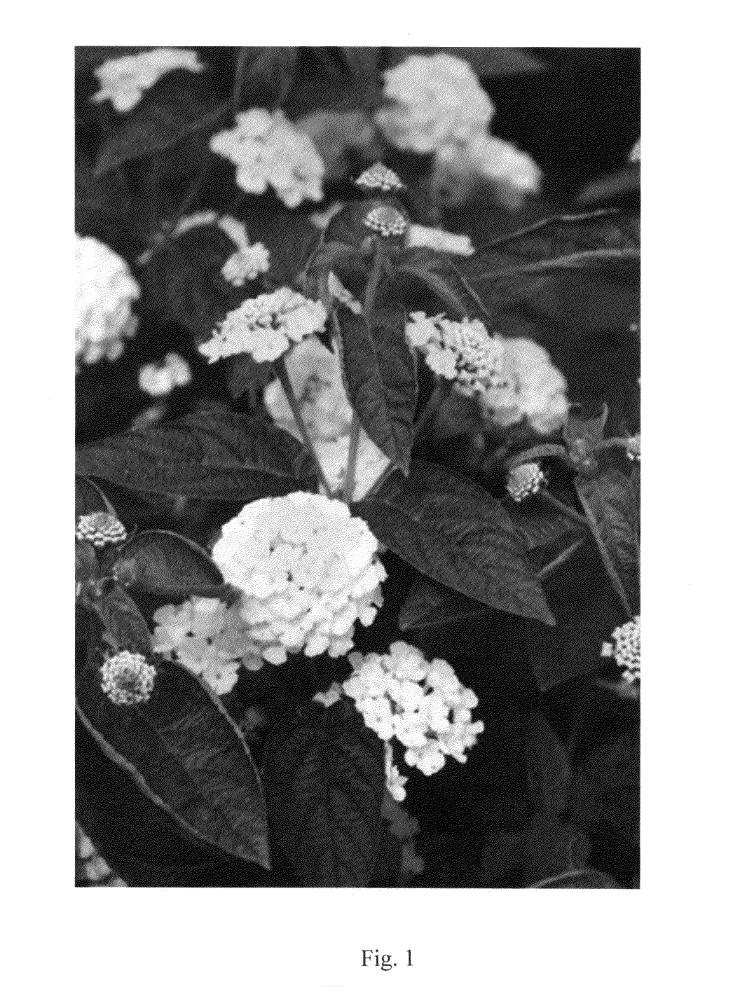

FIG. 2 is a close up photograph of the flowers of the new variety.

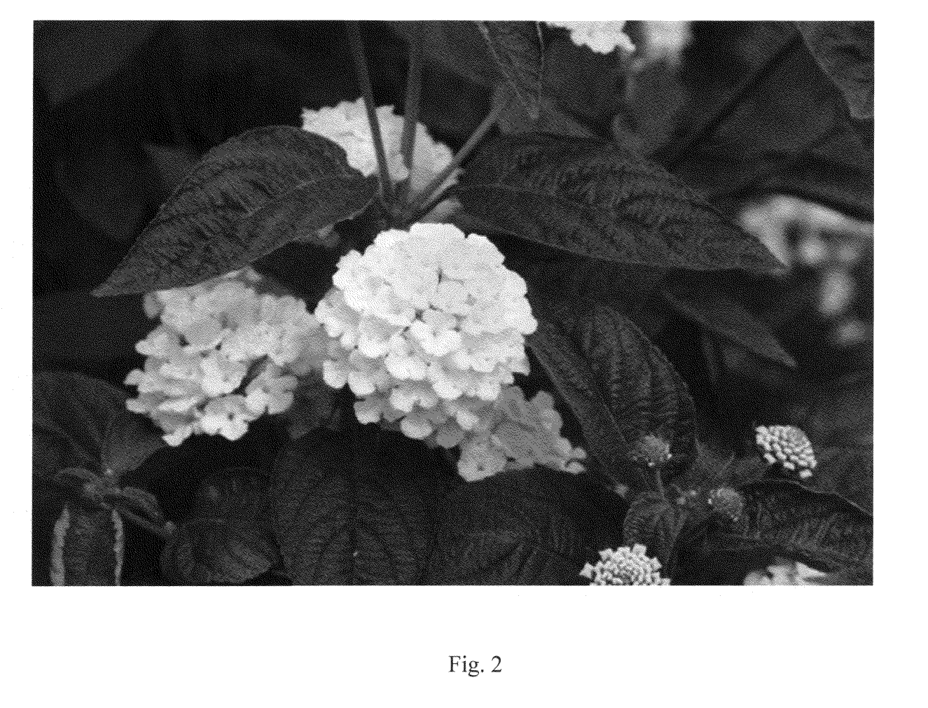

### DESCRIPTION OF THE PLANT

The following detailed description sets forth the characteristics of the new cultivar. The data which defines these characteristics were collected by asexual reproductions carried out in Muenster, Germany. The new variety was grown in a greenhouse under natural light at temperatures of 16-18° C. Color readings were taken under natural light. The new variety was 15 weeks old and grown in a 12 cm container when described. Color references are primarily to the R.H.S. Colour Chart of The Royal Horticultural Society of London.
Plant:
   Time to initiate roots.—About 10 days at about 20° C.
   Time to develop roots.—About 20 days at about 18-20° C.
   Time to produce a finished flowering plant from a rooted cutting.—About 10 weeks in a 12 cm container.
   Rooting habit.—Shallow, with good branching.
   Form.—Mounded.
   Height from media surface to top of foliage.—25 cm.
   Height from media surface to top of flowers.—25 cm.
   Plant diameter.—30 cm.
   Vigor.—Strong.
   Branching habit.—Medium to strong.
   Lateral branch.—Length: 5 cm. Diameter: 0.3-0.5 cm. Internode length: 5 cm. Strength: Upright and strong. Texture: Hispid. Color: Green, turning brown as the plant ages.
   Foliage.—Arrangement: Single and opposite. Length 6-7 cm. Width: 3.5-4 cm. Shape of leaf: Lanceolate. Apex: Acute. Base: Round and pointed. Margin: Serrate. Texture (both surfaces): Rough; pilary; hispid. Aspect: Horizontal. Pubescence (both surfaces): Light. Color: Young leaves: Upper surface: Green. Lower surface: Light green. Mature leaves: Upper surface: Dark green. Lower surface: Green. Petiole: Length: 5-6 cm. Diameter: 0.2 cm. Texture: Pilary; hispid. Color: Green. Veins: Venation type: Feather. Color Upper surface: Green. Lower surface: Green.
Flowers:
   Flower arrangement.—Simple; umbrella.
   Flower habit.—Trumpet-like.
   Natural flowering season.—Spring until later summer (April until frost).
   Fragrance.—Lemon balm.
   Inflorescence.—Height: 2 cm. Diameter: 3-4 cm.
   Buds (described 1-2 days before opening).—Length: 0.6 cm. Diameter: 0.2 cm. Shape: Trumpet-like. Color: Cream yellow-green.
   Flowers.—Appearance: Small balls made up of 40-50 flowers. Diameter: 1-1.2 cm. Height: 1.5 cm. Shape: Trumpet-like. Throat diameter: 0.1-0.2 cm. Tube: Length: 1.2-1.3 cm. Diameter: 0.2 cm.
   Corolla.—Arrangement: Base is fused to the tube. Petals: Color: Upper surface (when opening): 4A. Upper surface (fully opened): 4A. Lower surface (when opening): 7D-4D. Lower surface (fully opened): N999D. Shape: Truncate. Length: 2 cm. Width: 0.5 cm. Apex: Truncate and slightly unrelated. Base: Fused to tube. Margin: Smooth. Number per whorl: 5. Texture: Smooth. Throat color: Cream-yellow. Tube color: Cream-yellow.
   Calyx.—Appearance: Fused to small tube. Length: 0.3 cm. Diameter: 0.1 cm. Sepals: Apex: Pointy. Base: Fused to tube. Texture: Pubescence present. Margin: Pubescence present. Color (both surfaces): Green.
   Peduncles.—None present.
   Umbrella stem: Length: 4-5 cm. Diameter: 0.2 cm. Texture: Pubescence present. Color: Green.
   Disease/pest resistance.—None observed to date.
   Temperature tolerance.—Medium.
   Drought tolerance.—Medium.
Reproductive organs:
   Stamens.—Number (per flower): 5. Filament: Length: 0.1 cm. Color: White. Anthers: Shape: Didynamous, bithecous. Length: 0.1 cm. Color: Yellow-white. Pollen: Color: Yellow. Amount: Medium.
   Pistils.—Number (per flower): 1. Length: 0.3-0.4 cm. Style: Length: 0.3 cm. Color: White. Stigma: Shape: Obliquely subcapitate. Size: 0.2 cm. Ovaries: Position: Superior. Length: 0.1 cm. Width: 0.1 cm. Color: Green.
   Seeds.—Number: 1. Length: 0.5-0.6 cm. Diameter: 0.4 cm. Color: Dark brown-black.

I claim:

1. A new and distinct variety of lantana plant named ‘NEBLAN5151’ as is herein described and illustrated.
